(19) United States
(12) Patent Application Publication
(10) Pub. No.: US 2011/0178137 A1
(43) Pub. Date: Jul. 21, 2011

(54) DIARYL UREA FOR TREATING HEART FAILURE

(75) Inventors: Barbara Albrecht-Küpper, Wulfrath (DE); Stefan Schäfer, Goch (DE); Elodie Kienlen, Hattingen (DE)

(73) Assignee: BAYER SCHERING PHARMA AKTIENGESELLSCHAFT, Berlin (DE)

(21) Appl. No.: 13/001,193

(22) PCT Filed: Jun. 16, 2009
(86) PCT No.: PCT/EP2009/004303

§ 371 Date: Apr. 1, 2011

(30) Foreign Application Priority Data
Jun. 25, 2008 (EP) 08011474.7

(51) Int. Cl. A61K 31/44; C07D 213/78; A61P 9/00

(52) U.S. Cl. 514/350; 546/298

(57) ABSTRACT

The present invention relates to pharmaceutical compositions and combinations for treating, preventing or managing heart failure and/or connected diseases therewith comprising 4{4-[3-(4-chloro-3-trifluoromethylphenyl)-ureido]-3-fluorophenoxy}-pyridine-2-carboxylic acid methylamide optionally combined with at least one additional therapeutic agent.

The present invention relates to pharmaceutical compositions and combinations for treating, preventing or managing heart failure and/or connected diseases therewith comprising 4{4-[3-(4-chloro-3-trifluoromethylphenyl)-ureido]-3-fluorophenoxy}-pyridine-2-carboxylic acid methylamide optionally combined with at least one additional therapeutic agent.

Diaryl urea compounds e.g. 4{4-[3-(4-chloro-3-trifluoromethylphenyl)-ureido]-3-fluorophenoxy}-pyridine-2-carboxylic acid methylamide as described e.g. in US 20050038080 are potent anti-cancer and anti-angiogenic agents that possess various activities, including inhibitory activity on the VEGFR, PDGFR, raf, p38, and/or flt-3 kinase signaling molecules. These diaryl urea compounds have been previously characterized as having various activities, including for inhibiting the Raf/MEK/ERK pathway, raf kinase, p38 kinase, VEGFR kinase, PDGFR kinase. These activities and their use in treating various diseases and conditions are disclosed in, e.g., WO 2005/009961.

Chronic heart failure (CHF) is a clinical syndrome associated with an ominous long-term prognosis and major economic consequences for Western societies. There are over 15-18 million CHF patients in the US and Europe (Bonney. Cardiovascular disease therapeutics: market outlook 2004-2008. SCRIP Reports, PJB Publications Ltd, 2004).

Despite important progress made in its management in the last decade, heart failure (HF) remains associated with a long-term prognosis of 50% of patients dying within 4 years of diagnosis. The main medical cause for the development of CHF is coronary heart disease in 54-70% of patients. In the majority of cases this development is triggered by an initial ischemic event, like myocardial infarction. The continued aging of the population and more patient surviving acute myocardial infarction contribute to growing prevalent population prevalence of 1-2%. The second main cause of heart failure development is due to long lasting hypertension in patients.

Several types of drugs have proven useful in the treatment of heart failure like β-blockers, diuretics, ACE inhibitors, ATII antagonists and Aldosterone inhibitors.

The present invention provides pharmaceutical compositions for treating, preventing or managing heart failure and/or connected diseases therewith comprising a compound of formula I and optionally at least one further therapeutic agent.

The present invention can be used e.g. by administering a diaryl urea compound of formula I and optionally a further therapeutic agent, pharmaceutically-acceptable salts thereof, and derivatives thereof, etc.

The compounds with the structure of formula I, pharmaceutically acceptable salts, polymorphs, solvates, hydrates metabolites and prodrugs thereof, including diastereoisomeric forms (both isolated stereoisomers and mixtures of stereoisomers) are collectively referred to herein as the “compounds of formula I”.

Formula (I) is as follows:

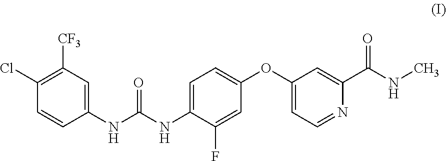

Where the plural form of the word compounds, salts, and the like, is used herein, this is taken to mean also a single compound, salt, or the like.

The present invention also relates to useful forms of the compounds as disclosed herein, such as pharmaceutically acceptable salts, metabolites and prodrugs. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, mangnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecyl-sulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoromethanesulfonate, and undecanoate.

Base salts include alkali metal salts such as potassium and sodium salts, alkaline earth metal salts such as calcium and magnesium salts, and ammonium salts with organic bases such as dicyclo-hexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aryl or aralkyl halides like benzyl and phenethyl bromides and others monosubstituted aralkyl halides or polysubstituted aralkyl halides.

Solvates for the purposes of the invention are those forms of the compounds where solvent molecules form a complex in the solid state and include, but are not limited to for example ethanol and methanol. Hydrates are a specific form of solvates, where the solvent molecule is water.

Certain pharmacologically active agents can be further modified with labile functional groups that are cleaved after in vivo administration to furnish the parent active agent and the pharmacologically inactive derivatizing group. These derivatives, commonly referred to as prodrugs, can be used, for example, to alter the physicochemical properties of the active agent, to target the active agent to a specific tissue, to alter the pharmacokinetic and pharmacodynamic properties of the active agent, and to reduce undesirable side effects. Prodrugs of the invention include, e.g., the esters of appropriate compounds of this invention that are well-tolerated, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

Methods which can be used to synthesize other prodrugs are described in the following reviews on the subject, which are incorporated herein by reference for their description of these synthesis methods:
Higuchi, T.; Stella, V. eds. Prodrugs As Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: New York (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff, M. E. ed. Burgers Medicinal Chemistry And Drug Discovery John Wiley & Sons: New York (1997), 949-982.

The metabolites of the compounds of this invention include oxidized derivatives of the compounds of formula I, wherein one or more of the nitrogens are substituted with a hydroxy group; which includes derivatives where the nitrogen atom of the pyridine group is in the oxide form, referred to in the art as 1-oxo-pyridine or has a hydroxy substituent, referred to in the art as 1-hydroxy-pyridine.

General Preparative Methods

The compounds of the invention may be prepared by use of known chemical reactions and procedures as described e.g. in the following published international application WO 2005/009961.

Further Therapeutic Agents

The compounds according to the invention can be used alone or if necessary in combination with further therapeutic agents. A further object of the present invention are medicaments which contain at least one of the compounds according to the invention and one or more further therapeutic agents, in particular for the treatment and/or prophylaxis of the diseases mentioned above and below. As combination active substances suitable for this, the following may for example and preferably be mentioned:
organic nitrates and NO donors, such as for example sodium nitroprusside, nitroglycerine, isosorbide mononitrate, isosorbide dinitrate, molsidomine or SIN-1, and inhalational NO;
diuretics, in particular loop diuretics and thiazides and thiazide-like diuretics;
positive-inotropically active compounds, such as for example cardiac glycosides (digoxin), and beta-adrenergic and dopaminergic agonists such as isoproterenol, adrenalin, noradrenalin, dopamine and dobutamine;
compounds which inhibit the degradation of cyclic guanosine monophosphate (cGMP) and/or cyclic adenosine monophosphate (cAMP), such as for example inhibitors of phosphodiesterases (PDE) 1, 2, 3, 4 and/or 5, in particular PDE 5 inhibitors such as sildenafil, vardenafil and tadalafil, and PDE 3 inhibitors such as amrinone and milrinone;
natriuretic peptides such as for example “atrial natriuretic peptide” (ANP, anaritide), “B-type natriuretic peptide” or “brain natriuretic peptide” (BNP, nesiritide), “C-type natriuretic peptide” (CNP) and urodilatin;
calcium sensitisers, such as for example and preferably levosimendan;
NO- and haem-independent activators of guanylate cyclase, such as in particular the compounds described in WO 01/19355, WO 01/19776, WO 01/19778, WO 01/19780, WO 02/070462 and WO 02/070510;
NO-independent, but haem-dependent stimulators of guanylate cyclase, such as in particular the compounds described in WO 00/06568, WO 00/06569, WO 02/42301 and WO 03/095451;
Inhibitors of human neutrophil elastase (HNE), such as for example sivelestat or DX-890 (reltran);
Compounds inhibiting the signal transduction cascade, such as for example tyrosine kinase inhibitors, in particular imatinib, gefitinib and erlotinib;
compounds influencing the energy metabolism of the heart, such as for example and preferably etomoxir, dichloracetate, ranolazine or trimetazidine;
agents with antithrombotic action, for example and preferably from the group of the thrombocyte aggregation inhibitors, anticoagulants or profibrinolytic substances;
blood pressure-lowering active substances, for example and preferably from the group of the calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists and rho-kinase inhibitors; and/or
active substances modifying fat metabolism, for example and preferably from the group of the thyroid receptor agonists, cholesterol synthesis inhibitors such as for example and preferably HMG-CoA reductase or squalene synthesis inhibitors, ACAT inhibitors, CETP inhibitors, MTP inhibitors, PPAR-alpha-, PPAR-gamma- and/or PPAR-delta agonists, cholesterol absorption inhibitors, lipase inhibitors, polymeric gallic acid adsorbers, gallic acid reabsorption inhibitors and lipoprotein(a) antagonists.

In a preferred embodiment of the invention, the compounds according to the invention are administered in combination with a diuretic, such as for example and preferably furosemid, bumetanid, torsemid, bendroflumethiazid, chlorthiazid, hydrochlorthiazid, hydroflumethiazid, methyclothiazid, polythiazid, trichlormethiazid, chlorthalidon, indapamid, metolazon, quinethazon, acetazolamid, dichlorophenamid, methazolamid, glycerine, isosorbide, mannitol, amilorid or triamteren.

Agents with antithrombotic action are understood preferably to mean compounds from the group of the thrombocyte aggregation inhibitors, anticoagulants or profibrinolytic substances.

In a preferred embodiment of the invention, the compounds according to the invention are administered in combination with a thrombocyte aggregation inhibitor, such as for example and preferably acetylsalicylic acid, clopidogrel, ticlopidine or dipyridamol.

In a preferred embodiment of the invention, the compounds according to the invention are administered in combination with a thrombin inhibitor, such as for example and preferably ximelagatran, melagatran, bivalirudin or clexane.

In a preferred embodiment of the invention, the compounds according to the invention are administered in combination with a GPIIb/IIIa antagonist, such as for example and preferably tirofiban or abciximab.

In a preferred embodiment of the invention, the compounds according to the invention are administered in combination with a factor Xa inhibitor, such as for example and preferably rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as for example and preferably coumarin.

Blood pressure-lowering agents are understood preferably to mean compounds from the group of the calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists, rho-kinase inhibitors and diuretics.

In a preferred embodiment of the invention, the compounds according to the invention are administered in combination with a calcium antagonist, such as for example and preferably nifedipin, amlodipin, verapamil or diltiazem.

In a preferred embodiment of the invention, the compounds according to the invention are administered in combination with an angiotensin AII antagonist, such as for example and preferably losartan, candesartan, valsartan, telmisartan or embusartan.

In a preferred embodiment of the invention, the compounds according to the invention are administered in combination with an ACE inhibitor, such as for example and preferably enalapril, captopril, lisinopril, ramipril, delapril, fosinopril, quinopril, perindopril or trandopril.

In a preferred embodiment of the invention, the compounds according to the invention are administered in combination with a vasopeptidase inhibitor or inhibitor of neutral endopeptidase (NEP), such as for example and preferably omapatrilat or AVE-7688.

In a preferred embodiment of the invention, the compounds according to the invention are administered in combination with an endothelin antagonist, such as for example and preferably bosentan, darusentan, ambrisentan or sitaxsentan.

In a preferred embodiment of the invention, the compounds according to the invention are administered in combination with a renin inhibitor, such as for example and preferably aliskiren, SPP-600 or SPP-800.

In a preferred embodiment of the invention, the compounds according to the invention are administered in combination with an alpha-1 receptor blocker, such as for example and preferably prazosin.

In a preferred embodiment of the invention, the compounds according to the invention are administered in combination with a beta receptor blocker, such as for example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with a mineralocorticoid receptor antagonist, such as for example and preferably spironolactone, eplerenon, canrenon or potassium canrenoate.

In a preferred embodiment of the invention, the compounds according to the invention are administered in combination with a rho-kinase inhibitor, such as for example and preferably fasudil, Y-27632, SLx-2119, BF-66851, BF-66852, BF-66853, KI-23095 or BA-1049.

Fat metabolism-modifying agents are understood preferably to mean compounds from the group of the CETP inhibitors, thyroid receptor agonists, cholesterol synthesis inhibitors such as HMG-CoA reductase or squalene synthesis inhibitors, ACAT inhibitors, MTP inhibitors, PPAR-alpha-, PPAR-gamma- and/or PPAR-delta agonists, cholesterol absorption inhibitors, polymeric gallic acid adsorbers, gallic acid reabsorption inhibitors, lipase inhibitors and lipoprotein(a) antagonists.

In a preferred embodiment of the invention, the compounds according to the invention are administered in combination with a CETP inhibitor, such as for example and preferably torcetrapib (CP-529 414), JJT-705, BAY 60-5521, BAY 78-7499 or CETP-vaccine (avant).

In a preferred embodiment of the invention, the compounds according to the invention are administered in combination with a thyroid receptor agonist, such as for example and preferably D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of the statins, such as for example and preferably lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, such as for example and preferably BMS-188494 or TAK-475.

In a preferred embodiment of the invention, the compounds according to the invention are administered in combination with an ACAT inhibitor, such as for example and preferably avasimibe, melinamide, pactimibe, eflucimibe or SMP-797.

In a preferred embodiment of the invention, the compounds according to the invention are administered in combination with an MTP inhibitor, such as for example and preferably implitapide, BMS-201038, R-103757 or JTT-130.

In a preferred embodiment of the invention, the compounds according to the invention are administered in combination with a PPAR-gamma agonist, such as for example and preferably pioglitazone or rosiglitazone.

In a preferred embodiment of the invention, the compounds according to the invention are administered in combination with a PPAR-delta agonist, such as for example and preferably GW-501516 or BAY 68-5042.

In a preferred embodiment of the invention, the compounds according to the invention are administered in combination with a cholesterol absorption inhibitor, such as for example and preferably ezetimibe, tiqueside or pamaqueside.

In a preferred embodiment of the invention, the compounds according to the invention are administered in combination with a lipase inhibitor, such as for example and preferably orlistat.

In a preferred embodiment of the invention, the compounds according to the invention are administered in combination with a polymeric gallic acid adsorber, such as for example and preferably cholestyramine, colestipol, colesolvam, cholestagel or colestimid.

In a preferred embodiment of the invention, the compounds according to the invention are administered in combination with a gallic acid reabsorption inhibitor, such as for example and preferably ASBT (=IBAT) inhibitors such as for example AZD-7806, S-8921, AK-105, BARI-1741, SC-435 or SC-635.

In a preferred embodiment of the invention, the compounds according to the invention are administered in combination with a lipoprotein(a) antagonist, such as for example and preferably gemcabene calcium (CI-1027) or nicotinic acid.

Indications

The compounds and combinations according to the invention can be used for manufacture of a medicament for the prophylaxis and/or treatment of heart failure and connected diseases therewith. Also the present invention provides methods of treating, preventing and managing heart failure and/or connected diseases therewith, comprising administering effective amounts of at least one compound of formula I and optionally at least one further therapeutic agent according to the invention. An “effective amount” is the quantity of the compound that is useful to achieve the desired result, e.g., to treat, prevent or manage the disease or condition. In this connection, the following may for example and preferably be mentioned as target indications: acute and chronic cardiac insufficiency, arterial hypertension, coronary heart disease, stable and unstable angina pectoris, myocardial ischemia, myocardial infarction, shock, arteriosclerosis, atrial and ventricular arrhythmias, transitory and ischemic attacks, stroke, inflammatory cardiovascular diseases, peripheral and cardiac vascular diseases, peripheral circulation disorders, spasms of the coronary arteries and peripheral arteries, thromboses, thromboembolic diseases, edema formation such as for example pulmonary edema, cerebral edema, renal edema or cardiac insufficiency-related edema, and restenosis for example after thrombolysis treatments, percutaneous-transluminal angioplasties (PTA), transluminal coronary angioplasties (PTCA), heart transplants and bypass operations.

In the sense of the present invention, the term cardiac insufficiency also includes more specific or related disease forms such as right cardiac insufficiency, left cardiac insufficiency, global insufficiency, ischemic cardiomyopathy, dilatative cardiomyopathy, congenital heart defects, heart valve defects, cardiac insufficiency with heart valve defects, mitral valve stenosis, mitral valve insufficiency, aortic valve stenosis, aortic valve insufficiency, tricuspidal stenosis, tricuspidal insufficiency, pulmonary valve stenosis, pulmonary valve insufficiency, combined heart valve defects, heart muscle inflammation (myocarditis), chronic myocarditis, acute myocarditis, viral myocarditis, diabetic cardiac insufficiency, alcohol-toxic cardiomyopathy, cardiac storage diseases, diastolic cardiac insufficiency and systolic cardiac insufficiency.

According to the invention the term “treating” refers to the administration of a pharmaceutical composition after the onset of symptoms, whereas “preventing” refers to the administration prior to the onset of symptoms, particularly to patients at risk. The term “managing” encompasses preventing the recurrence of a disease in a patient who suffered from that disease.

Administration

Compounds or drug combinations of the present invention can be administered in any form by any effective route, including, e.g., oral, parenteral, enteral, intravenous, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive.

Preference is given to an oral administration.

Compounds or drug combinations of the present invention can be converted in a known manner into the usual formulations, which may be liquid or solid formulations e.g. without limitation normal and enteric coated tablets, capsules, pills, powders, granules, elixirs, tinctures, solution, suspensions, syrups, solid and liquid aerosols and emulsions.

Examples of solid formulations for oral administration are described in U.S. provisional application No. 60/605,752.

The combinations of the present invention can be administered at any time and in any effective form. For example, the compounds can be administered simultaneously, e.g., as a single composition or dosage unit (e.g., a pill or liquid containing both compositions), or they can be administered as separate compositions, but at the same time (e.g., where one drug is administered intravenously and the other is administered orally or intramuscularly). The drugs can also be administered sequentially at different times. Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The drug combinations can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy.

Compounds or drug combinations of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

In addition, compounds or drug combinations of the present invention can be administered with other active agents or other therapies that are utilized to treat any of the above-mentioned diseases and/or conditions.

Other therapies according to the invention include, but are not limited to, e.g. physical or mechanical therapy such as electrical stimulation, acupuncture, magnet therapy or topical use of polyurethane films.

The present invention provides also combinations of at least one compound of Formula I and at least one other therapeutic agent mentioned above useful in treating a disease or disorder. “Combinations” for the purposes of the invention include:
single compositions or dosage forms which contain at least one compound of Formula I and at least one other therapeutic agent mentioned above;
combination packs containing at least one compound of Formula I and at least one other therapeutic agent mentioned above to be administered concurrently or sequentially;
kits which comprise at least one compound of Formula I and at least one other therapeutic agent mentioned above packaged separate from one another as unit dosages or as independent unit dosages, with or without instructions that they be administered concurrently or sequentially; and
separate independent dosage forms of at least one compound of Formula I and at least one other therapeutic agent mentioned above which cooperate to achieve a therapeutic effect, e.g., treatment of the same disease, when administered concurrently or sequentially.

The dosage of each agent of the combination can be selected with reference to the other and/or the type of disease and/or the disease status in order to provide the desired therapeutic activity. For example, the active agents in the combination can be present and administered in a fixed combination. “Fixed combination” is intended here to mean pharmaceutical forms in which the components are present in a fixed ratio that provides the desired efficacy. These amounts can be determined routinely for a particular patient, where various parameters are utilized to select the appropriate dosage (e.g., type of disease, age of patient, disease status, patient health, weight, etc.), or the amounts can be relatively standard.

The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

Preference is given to an amount of the compound of formula I from 20 to 2000 mg, preferably from 40 to 800 mg, more preferably from 50 to 600 mg.

Particular preference is given to an amount of 4{4-[3-(4-chloro-3-trifluoromethylphenyl)-ureido]-3-fluorophenoxy}-pyridine-2-carboxylic acid methylamide in the pharmaceutical composition from 20 to 3000 mg, preferably from 50 to 1500, more preferably from 60 to 1000 mg.

In another embodiment of the invention the compound of formula I is administered in combination with at least one further therapeutic agent in an amount that those of ordinary skill in the art can determine by their professional judgement.

The pharmaceutical composition according to the invention is administered one or more, preferably up to three, more preferably up to two times per day. Preference is given to an administration via the oral route. With each administration the number of tablets or capsules taken in at the same time should not exceed two.

Nevertheless, it may in some cases be advantageous to deviate from the amounts specified, depending on body weight, individual behaviour toward the active ingredient, type of preparation and time or interval over which the administration is effected. For instance, less than the aforementioned minimum amounts may be sufficient in some cases, while the upper limit specified has to be exceeded in other cases. In the case of administration of relatively large amounts, it may be advisable to divide these into several individual doses over the day.

The combination can comprise effective amounts of at least one compound of Formula I and at least one other therapeutic agent mentioned above, which achieves a greater therapeutic efficacy than when either compound is used alone.

The relative ratios of each compound in the combination can also be selected based on their respective mechanisms of action and the disease biology. The relative ratios of each compound can vary widely and this invention includes combinations where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising a compound of formula I and at least one compound selected from the group consisting of phosphodiesterase V inhibitors, endothelin antagonists, prostacyclin analogues, kinase inhibitors and elastase inhibitors. More preferably a combination comprising 4{4[3-(4-chloro-3-trifluoromethylphenyl)-ureido]-3-fluorophenoxy}-pyridine-2-carboxylic acid methylamide and and at least one compound selected from the group consisting of tadalafil, sildenafil, vardenafil, bosentan, sitaxentan, ilomedin, treprostinil and epoprostenol is used. Most preferably a combination comprising 4{4-[3-(4-chloro-3-trifluoromethylphenyl)-ureido]-3-fluorophenoxy}-pyridine-2-carboxylic acid methylamide and bosentan or vardenafil is used.

### EXAMPLE

The effects of the compound and drug combinations according to the invention are tested in vivo in mouse models of pressure-induced heart failure.

Methods: Pressure-Induced Heart Failure (TAC)

Surgery: 8 weeks old mice are pre-anesthetized for 2-3 min in an isofluran flooded box and then intubated (tubus of own fabrication).

Mice are placed on the right side on a heating panel and the tubus is connected to a ventilation pump (Mini Vent Type 845, Hugo Sachs Electronic) which allows the further ventilation of mice with isofluran (1.5%).

Mice are prepared for the surgery: eyes moistened with Bepanthen® salve (pharmaceutical ointment containing dexpanthenol), operation field shaved and disinfected, mice fixated with tape in a right lateral position.

The skin cut is placed ca 2 mm behind the left elbow and is 5 mm length and vertical. Pectoral muscles are separated until ribs. A wound-spreader is placed between the 2^{nd} and the 3^{rd} rib and thymus is visualized. After pushing by side the thymus V. cava, A. pulmonalis and Aorta with both carotid branching are viewed. With help from a curved polished vessel catheter a ligation thread is placed around the aorta between both carotid branching. The aorta is constricted to a diameter of 0.6 mm with help from a splint.

By sham operated mice the same procedure is effectuated but the aorta is not constricted.

Before they woke up, mice are given 5 mg/5 ml/kg sc Rimadyl® (Pfizer, Carprofen), wound is cleaned with 9% NaCl and coated with antibiotic salve (Neomycin® salve) and Bepanthen® salve is removed from the eyes.

After awaking from anesthesia the tubus is taken off and mice are placed in their cages heated with heating panels for at least half an hour.

Mice are divided into 3 groups (n=10-12):
sham operated with vehicle treatment
placebo with vehicle treatment
4{4-[3-(4-chloro-3-trifluoromethylphenyl)-ureido]-3-fluorophenoxy}-pyridine-2-carboxylic acid methylamide

Substances are dissolved in 10% Ethanol, 40% Solutol and 50% water.

Application volumes: 10 ml/kg/d po

Treatment: begin 1 day after surgery for 4 weeks

After 4 weeks of treatment hemodynamic measurement are performed and mice are sacrificed.

Hemodynamic measurements: Mice are pre-anesthetized for 2-3 min in an isofluran flooded box, followed by fixation in an anesthesia mask (continuous flow of 1.5% isofluran by spontaneous breathing of the mouse). Mice are placed on the back on a heating panel.

Throats are shaved and the skin incised on the median line. The right A. carotis is prepared and ligated cranial. A tip-catheter (Millar Micro-Tip-Transducer, 1.0 oder 1.4 French, Firma HSE) is introduced in the right A. carotis and pushed into the left ventricle and fixated. There, pressure of the left ventricle is registered for a few minutes (after reaching a steady state) over the catheter and analyzed by the Millar Chart 5 software. After the measurements blood samples are taken. Finally organs (heart, lung, liver, right kidney) are collected.

### Example 1

### Preparation of a 4:1 co-precipitate formulationsolid dispersion of 4{4-[3-(4-chloro-3-trifluoromethylphenyl)-ureido]-3-fluorophenoxy}-pyridine-2-carboxylic acid methyl amide with polyvinylpyrrolidone

In an uncapped vial, one part of 4{4-[3-(4-(4-chloro-3-trifluoromethylphenyl)-ureido]-3-fluorophenoxy}-pyridine-2-carboxylic acid methyl amide as a free base was mixed with four parts polyvinylpyrrolidone (PVP-25/Kollidon 25), and dissolved in a sufficient amount of a 1:1 mixture of acetone and ethanol, until all powders are in solution. The uncapped vial was placed into a vacuum oven set at 40° C., and let dry for at least 24-48 hours.

What we claim:

1. Use of a compound of formula I or a pharmaceutically acceptable salt, polymorph, solvate, hydrate, metabolite, prodrug or diastereoisomeric form thereof, for manufacture of a medicament for treating, preventing or managing of heart failure and/or connected diseases therewith,
wherein said compound of formula I is:

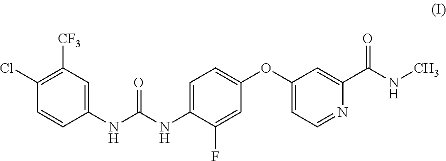

2. Use of claim 1 wherein the treating, preventing or managing of heart failure and/or connected diseases therewith is selected from the group consisting of acute and chronic cardiac insufficiency, arterial hypertension, coronary heart disease, stable and unstable angina pectoris, myocardial ischemia, myocardial infarction, shock, arteriosclerosis, atrial and ventricular arrhythmias, transitory and ischemic attacks, stroke, inflammatory cardiovascular diseases, peripheral and cardiac vascular diseases, peripheral circulation disorders, spasms of the coronary arteries and peripheral arteries, thromboses, thromboembolic diseases, edema formation such as for example pulmonary edema, cerebral edema, renal edema or cardiac insufficiency-related edema, and restenosis for example after thrombolysis treatments, percutaneous-transluminal angioplasties (PTA), transluminal coronary angioplasties (PTCA), heart transplants and bypass operations.

3. Use of claim 1 wherein the treating, preventing or managing of heart failure and/or connected diseases therewith is selected from the group consisting of right cardiac insufficiency, left cardiac insufficiency, global insufficiency, ischemic cardiomyopathy, dilatative cardiomyopathy, congenital heart defects, heart valve defects, cardiac insufficiency with heart valve defects, mitral valve stenosis, mitral valve insufficiency, aortic valve stenosis, aortic valve insufficiency, tricuspidal stenosis, tricuspidal insufficiency, pulmonary valve stenosis, pulmonary valve insufficiency, combined heart valve defects, heart muscle inflammation (myocarditis), chronic myocarditis, acute myocarditis, viral myocarditis, diabetic cardiac insufficiency, alcohol-toxic cardiomyopathy, cardiac storage diseases, diastolic cardiac insufficiency and systolic cardiac insufficiency.

4. Combination comprising at least one compound of formula I as defined in claim 1 and at least one further therapeutic agent selected from the group consisting of organic nitrates, NO donors, diuretics, positive-inotropically active compounds, compounds which inhibit the degradation of cyclic guanosine monophosphate (cGMP), cyclic adenosine monophosphate (cAMP), natriuretic peptides, calcium sensitisers, NO- and haem-independent activators of guanylate cyclase, NO-independent, but haem-dependent stimulators of guanylate cyclase, inhibitors of human neutrophil elastase (HNE), compounds inhibiting the signal transduction cascade, compounds influencing the energy metabolism of the heart, agents with antithrombotic action, blood pressure-lowering active substances, active substances modifying fat metabolism, thrombocyte aggregation inhibitors, anticoagulants, profibrinolytic substances, GPIIb/IIIa antagonist, factor Xa inhibitor, thrombin inhibitor, heparin or a low molecular weight (LMW) heparin derivative, vitamin K antagonist, calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists, rho-kinase inhibitors, HMG-CoA reductase or squalene synthesis inhibitors, ACAT inhibitors, MTP inhibitors, PPAR-alpha-, PPAR-gamma- and/or PPAR-delta agonists, cholesterol absorption inhibitors, polymeric gallic acid adsorbers, gallic acid reabsorption inhibitors, lipase inhibitors and lipoprotein(a) antagonists, CETP inhibitors, thyroid receptor agonists and cholesterol synthesis inhibitors.

5. Pharmaceutical composition comprising a combination as defined in claim 4.

6. Pharmaceutical composition of claim 5 for treating, preventing or managing of heart failure and/or connected diseases therewith.

7. Pharmaceutical composition of claim 6 wherein the treating, preventing or managing of heart failure and/or connected diseases therewith is selected from the group consisting of acute and chronic cardiac insufficiency, arterial hypertension, coronary heart disease, stable and unstable angina pectoris, myocardial ischemia, myocardial infarction, shock, arteriosclerosis, atrial and ventricular arrhythmias, transitory and ischemic attacks, stroke, inflammatory cardiovascular diseases, peripheral and cardiac vascular diseases, peripheral circulation disorders, spasms of the coronary arteries and peripheral arteries, thromboses, thromboembolic diseases, edema formation such as for example pulmonary edema, cerebral edema, renal edema or cardiac insufficiency-related edema, and restenosis for example after thrombolysis treatments, percutaneous-transluminal angioplasties (PTA), transluminal coronary angioplasties (PTCA), heart transplants and bypass operations.

8. Pharmaceutical composition of claim 6 wherein the treating, preventing or managing of heart failure and/or connected diseases therewith is selected from the group consisting of right cardiac insufficiency, left cardiac insufficiency, global insufficiency, ischemic cardiomyopathy, dilatative cardiomyopathy, congenital heart defects, heart valve defects, cardiac insufficiency with heart valve defects, mitral valve stenosis, mitral valve insufficiency, aortic valve stenosis, aortic valve insufficiency, tricuspidal stenosis, tricuspidal insufficiency, pulmonary valve stenosis, pulmonary valve insufficiency, combined heart valve defects, heart muscle inflammation (myocarditis), chronic myocarditis, acute myocarditis, viral myocarditis, diabetic cardiac insufficiency, alcohol-toxic cardiomyopathy, cardiac storage diseases, diastolic cardiac insufficiency and systolic cardiac insufficiency.

9. A method for treating, preventing or managing of heart failure and/or connected diseases therewith in a subject in need thereof comprising administering effective amounts of a compound of formula I or a pharmaceutically acceptable salt, polymorph, solvate, hydrate, metabolite, prodrug or diastereoisomeric form thereof
wherein said compound of formula I is:

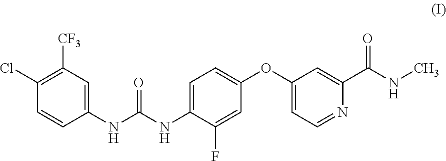

10. The method of claim 9 wherein the compound of formula I is combined with at least one further therapeutic agent selected from the group consisting of organic nitrates, NO donors, diuretics, positive-inotropically active compounds, compounds which inhibit the degradation of cyclic guanosine monophosphate (cGMP), cyclic adenosine monophosphate (cAMP), natriuretic peptides, calcium sensitisers, NO- and haem-independent activators of guanylate cyclase, NO-independent, but haem-dependent stimulators of guanylate cyclase, inhibitors of human neutrophil elastase (HNE), compounds inhibiting the signal transduction cascade, compounds influencing the energy metabolism of the heart, agents with antithrombotic action, blood pressure-lowering active substances, active substances modifying fat metabolism, thrombocyte aggregation inhibitors, anticoagulants, profibrinolytic substances, GPIIb/IIIa antagonist, factor Xa inhibitor, thrombin inhibitor, heparin or a low molecular weight (LMW) heparin derivative, vitamin K antagonist, calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists, rho-kinase inhibitors, HMG-CoA reductase or squalene synthesis inhibitors, ACAT inhibitors, MTP inhibitors, PPAR-alpha-, PPAR-gamma- and/or PPAR-delta agonists, cholesterol absorption inhibitors, polymeric gallic acid adsorbers, gallic acid reabsorption inhibitors, lipase inhibitors and lipoprotein(a) antagonists, CETP inhibitors, thyroid receptor agonists and cholesterol synthesis inhibitors.
